(19) United States
(12) Patent Application Publication
(10) Pub. No.: US 2012/0142104 A1
(43) Pub. Date: Jun. 7, 2012

(54) SUPPLEMENT FOR CELL CULTIVATION MEDIA

(76) Inventors: Yung-Kai Lin, Taipei (TW); Wei-Hsun You, Taipei County (TW)

(21) Appl. No.: 13/175,119

(22) Filed: Jul. 1, 2011

(30) Foreign Application Priority Data
Dec. 3, 2010 (TW) 099142184

(51) Int. Cl. C12N 5/071

(52) U.S. Cl. 435/408

(57) ABSTRACT

A method of preparing a supplement for cell cultivation media, which comprises concentrating porcine blood by centrifugation and obtaining supernatant, adding agonist for activating platelets into the supernatant to obtain activated supernatant, and sterilizing the activated supernatant is presented. A supplement for cell cultivation media, which is made by the above method, a cell cultivation media comprising the supplement, and a use of the cell cultivation media in culturing or treating cells in tissue engineering or regenerative medicine is also presented.

### FIELD OF THE INVENTION

The present invention relates to a new supplement for cell cultivation media.

### BACKGROUND OF THE INVENTION

Stem cell has become one main kind of the tissue engineering researches in recent years. It can differentiate into many kinds of tissue cells (e.g. osteocytes, neural cells, cardiomyocytes, and islet cells) which are able to be used in regenerative medicine. However, an obstacle must be conquered for clinical and tissue applications. It is difficult to achieve the medical demand by in vitro cell expansion in a short period. In this case, it is hard to become a common medical use.

Fetal bovine serum (FBS) is commonly applied as a supplement to culture medium in many researches. FBS is obtained by collecting blood from bovine fetus and removing blood colt which containing red blood cells and platelet by centrifugation. Cell expansion improvement is typically achieved by adding 5-20% FBS in culture medium. Using FBS as a supplement to culture medium has been a dispute. The production and application are accompanied with ethical and scientific controversies whole the time. The collecting process of FBS is generally achieved by harvesting blood from the umbilical cord with a catheter. Alternatively, the blood is harvested directly from the bovine fetus heart with a 12-16 gauge needle. Both two methods may cause pain to bovine fetus. The current annual global production of FBS is about 500,000 liters, and only 0.5 liters of serum can be obtained from each fetal bovine. It can be estimated that about one million fetal bovines are sacrificed for this each year. Therefore, the manufacture of FBS has been criticized by animal protection people. In science, serum is rich in proteins, growth factors, hormones, nutrients, metabolites, lipids, minerals and inhibitors. Its composition is complicated and has the possibility of the existence of contaminants. In this case, it often impact on the quality of research.

In Taiwan, about 2.5 liters of blood can be obtained from a pig which reached market weight (ranged from 95 kg to 105 kg). Thus, about 22 million liters of porcine blood should be collected for one year in Taiwan (The website of Council of Agriculture, Executive Yuan http://www.coa.gov.tw/show_index.php, 2009 livestock statistics). Currently, the application of porcine blood after collection within the territory of Taiwan are mainly related to processing of foods or feeds that made of spray-dried blood. Meanwhile the physiological structure and composition of porcine are similar to that of human, so the blood of porcine is able to accept applications. The blood product (BP) is a kind of treated platelet plasma concentrates. Its platelet concentration is about 8 times higher than whole blood. Each platelet contains about 50 to 80 α-granules. Platelet aggregates in injured tissues and stimulates α-granules to release growth factors under normal physiological clot reaction, in the purpose of wound healing. The α-granules may release several kinds of growth factors after activation, such as insulin-like growth factor (IGF), keratinocyte growth factor (KGF), platelet-derived growth (PDGF), and transforming growth factor (TGF-β).

### DETAILED DESCRIPTION OF THE INVENTION

The present invention develops a new supplement from porcine blood, which is adequate for culture medium. Porcine blood product (P-BP) is obtained by concentrating porcine blood with different centrifugation steps. Various agonist combination formulas are added to activate platelets, to release growth factors. After quantitative analysis, the optimal agonist formulas are designed by Design-Expert software. Heavy metal and fibronectin contents are also examined. The medium with the supplement is prepared, for the purpose of comparison between FBS medium and commercial serum free media. Comparison comprises their effect on adhesion, proliferation and survival rate of human mesenchymal stem cell. The present invention designs three groups: Co+T−Ca−, Co+T−Ca− and Co−T+Ca− as the optimal agonist formulas. The content analysis indicates that P-BP does not contain harmful heavy metals, and its fibronectin contents are less than commercial serum. At the same time, the result of cell culture shows slower adhesion rate. The result of cell culture shows that cell proliferation and survival rate can be improved by three kinds of P-BP. It can be seen that the nutritional supplements of culture medium in the present invention are more effective in improving the cell proliferation rate, and up to 2 times higher than the effect of commercial serum. The nutritional supplements of culture medium in the present invention are able to promote cell proliferation in a shorter period of time. Rapid growth of cells can be applied in tissue engineering or regenerating medicine. Comparing with commercial media, the nutritional supplements of culture medium in the present invention significantly reduce cell rupture and morphological damage and provide an optimum growth environment for cells. The nutritional supplements of culture medium in the present invention can also combine with biomedical materials, which can be placed in vivo for rapid wound healing effects thus contributes the development of the field of regenerative medicine.

The term “platelet activator” refers to substance stimulates platelets to release growth factor, said growth factor usually facilitate cell proliferation. The growth factors include but not limited to IGF (insulin-like growth factor) [symbol] KGF (keratinocyte growth factor) [symbol] PDGF (platelet-derived growth factor) and TGF-β (transforming growth factor-β).

The present invention provides a method for preparing a supplement for cell cultivation media, comprising: a) centrifuging mammal blood and collecting supernatant; b) adding platelets activator into the supernatant to obtain activation supernatant; and c) sterilizing the activation supernatant. In a preferred embodiment, the centrifuging of step a means centrifuging at 100-5000 g for three times; in a further preferred embodiment, the centrifuging of step a means centrifuging at 100-500 g for 5-10 minutes for the first time, centrifuging at 100-500 g for 5-10 minutes for the second time, and centrifuging at 1000-5000 g for 5-10 minutes for the third time. In another preferred embodiment, the supernatant of step to obtaining platelet concentration from 50000-100000 platelets/ml.

In a preferred embodiment, the platelet activators comprise agonists selected from a group consisting of collagen, thrombin and calcium chloride (CaCl₂) or alone. In a further preferred embodiment, the collagen, thrombin and CaCl₂ are in a range of concentration: collagen has concentration from 1000 μg/ml to 5000 μg/ml, thrombin has concentration from 5 U/ml to 50 U/ml, and CaCl₂ has concentration from 0.1% to 4.0%; collagen has concentration from 1000 μg/ml to 5000 μg/ml, thrombin has concentration from 0.1 U/ml to 2.0 U/ml, and CaCl₂ has concentration from 0.1% to 4.0%; or collagen has concentration from 10 μg/ml to 800 μg/ml, thrombin has concentration from 5 U/ml to 50 U/ml, and CaCl₂ has concentration from 0.1% to 4.0%.

In a preferred embodiment, the cell cultivation media is stem cell cultivation media.

In a preferred embodiment, the mammal is pig.

The present invention also provides a supplement for cell cultivation media made by the method of the invention. In a preferred embodiment, the supplement facilitates cell proliferation and cell survival. In another preferred embodiment, the supplement contains endotoxin less than 0.03 EU/mL. In further preferred embodiment, the supplement has inhibition activity of trypsin.

The present invention further provides a cell cultivation media contains the supplement made by the method of the invention, and the use of the cell cultivation media for culturing cells for tissue engineering, regenerative medicine or vaccine industry.

### EXAMPLES

The examples below are non-limiting and are merely representative of various aspects and features of the present invention.

### Example 1

### Preparation of Porcine Blood Product (P-BP)

1. A pig was slaughtered by cutting its carotid artery and allowing it to bleed out. The blood was collected into a centrifuge tube with anticoagulant, and kept in ice bath until lab process started.
2. The harvested blood was then centrifuged 100-5000 g three times at room temperature (first time at 100-500 g for 5-10 minutes, second time at 100-500 g for 5-10 minutes, and third time at 1000-5000 g for 5-10 minutes). The supernatant was collected.
3. Adding combination formula comprising collagen, thrombin, and calcium chloride at various concentrations into the supernatant. The concentration of collagen was 1000-5000 μg/ml in Co+ and 10-800 μg/ml in Co−. The concentration of thrombin was 5-50 U/ml in T+ and 0.1-2.0 U/ml in T−. The concentration of calcium chloride was 5-30% in Ca+ and 0.1-4.0% in Ca− (Table 1). The mixture was activated in a water bath at 15-45° C. while shaking at 50-100 rpm for 10-60 minutes. After activation, the activated supernatant was extracted by centrifugation at 500-15000 g.
4. The activated supernatant was filtered through 0.10-0.50 μm membrane to obtain the P-BP.

TABLE 1
Groups of agonist formulas

| | Col- | Throm- | | Col- | Throm- | | |
|---|---|---|---|---|---|---|---|
| | lagen | bin | CaCl₂ | lagen | bin | CaCl₂ | |
| | + | + | + | − | − | − | PBS |
| Co+ alone | + | | | | | | + + |
| T+ alone | | + | | | | | + + |
| Ca+ alone | | | + | | | | + + |
| Co−T+Ca+ | | + | + | + | | | |
| Co−T−Ca− | | | | + | + | + | |
| Co−T+Ca− | | + | | + | | | |
| Co+T−Ca− | + | | | | + | + | |
| Co+T+Ca+ | + | + | + | | | | |
| Co+T+Ca− | + | + | | | | + | |
| Co−T−Ca+ | | | + | + | + | | |
| Co+T−Ca+ | + | | + | | + | | |

### Example 2

### Quantitation of Growth Factors

1. Growth factor of P-BP was quantified using an ELISA kit (DG100, from R&D Systems). IGF content of P-BP was calculated against a standard curve.
2. Growth factor of P-BP was quantified using an ELISA kit (CSE-E06800, from CUSABIO). KGF content of P-BP was calculated against a standard curve.
3. Growth factor of P-BP was quantified using an ELISA kit (DHD00B, from R&D). PDGF content of P-BP was calculated against a standard curve.
4. Growth factor of P-BP was quantified using an ELISA kit (MB100B, from R&D systems). TGF-β content of P-BP was calculated against a standard curve.
5. The results showed that the releasing amounts of TGF-β, KGF, and PDGF in activated P-BP were significantly higher than in FBS (p<0.05), especially in TGF-β (p<0.05, see Table 2). The growth factors increased cell proliferation and shorten the time it required. The amounts of growth factors in P-BP were higher than in commercial FBS and serum free media. It will lead to faster cell proliferation and reach the medical or trail demand.

TABLE 2
The growth factor content of was measured by ELISA method

| | TGF-β (pg/mL) | IGF (ng/mL) | PDGF (pg/mL) | KGF (pg/mL) |
|---|---|---|---|---|
| FBS Brasil | 8396.37 ± 1119.40 | 67.65 ± 7.77* | 921.89 ± 449.28 | 84.43 ± 4.80 |
| FBS USA | 10707.49 ± 1525.08 | 59.28 ± 5.79 | 903.70 ± 445.59 | 90.14 ± 9.62 |
| (Hyclone) | | | | |
| Control | 11007.60 ± 1269.13 | 3.96 ± 5.00* | 822.15 ± 167.04 | 86.43 ± 1.43 |
| Co+ alone | 13622.27 ± 4147.37 | 29.25 ± 11.20* | 866.10 ± 219.48 | 101.90 ± 15.13 |
| T+ alone | 16108.15 ± 3219.14* | 27.19 ± 10.11* | 952.72 ± 417.96 | 104.52 ± 19.33 |
| Ca+ alone | 6495.83 ± 1900.91* | 28.68 ± 8.57* | 909.72 ± 366.68 | 100.48 ± 12.10 |
| Co−T+Ca+ | 7884.78 ± 1426.60* | 29.20 ± 8.36* | 896.46 ± 325.37 | 98.57 ± 13.05 |
| Co−T−Ca− | 15416.49 ± 4035.96 | 26.77 ± 9.47* | 943.32 ± 362.88 | 93.33 ± 17.13 |
| Co−T+Ca− | 15611.34 ± 1882.27 | 29.92 ± 7.74* | 1055.21 ± 480.50 | 100.95 ± 20.03 |
| Co+T−Ca− | 16815.71 ± 3398.05* | 28.77 ± 10.56* | 937.16 ± 289.42 | 100.48 ± 13.44 |
| Co+T+Ca+ | 6197.38 ± 2175.29* | 27.14 ± 11.84* | 907.41 ± 290.72 | 103.33 ± 9.49 |
| Co+T+Ca− | 12608.79 ± 2708.40* | 30.03 ± 14.82* | 991.72 ± 245.13 | 94.76 ± 7.08 |
| Co−T−Ca+ | 4974.41 ± 2819.41* | 30.15 ± 9.38* | 1143.71 ± 154.89 | 107.62 ± 17.29 |
| Co+T−Ca+ | 6081.66 ± 3357.81* | 28.66 ± 11.04* | 1160.81 ± 260.62 | 96.43 ± 14.17 |

### Example 3

### Designs of the Optimal Agonist Formulas:

The results of quantitation of growth factors were analyzed by Design-Expert software. The impact of the released growth factors due to each activator was calculated, and represented as ANOVA values (Table 3). A stranded for collagen, B stranded for thrombin, and C stranded for calcium chloride. ANOVA values indicated the impact of the released growth factors due to each activator. The larger value indicates that more growth factors were released from platelets due to the activator. The release of TGF-β and PDGF increased after collagen or calcium chloride activation. The secretion of KGF was increased due to calcium chloride. The results referred to RSM (response surface methodology) and Cube efficiency map (FIG. 1 to FIG. 8). Finally, the results showed that Co−T+Ca− [symbol] Co+T−Ca− and Co+T+Ca− would be optimal agonist formulas.

TABLE 3
Design-Expert software analysis

| TGF-β | | | PDGF | | |
|---|---|---|---|---|---|
| Final Equation in Terms of Coded Factors: | | | Final Equation in Terms of Coded Factors: | | |
| TGF-β = | +11791.39 | | PDGF = | +1065.51 | |
| | −370.46 | *A | | −8.86 | *A |
| | −749.84 | *B | | −26.80 | *B |
| | −4153.39 | *C | | +16.67 | *C |
| | −1104.07 | *A *B | | −18.93 | *A *B |
| | +274.91 | *A *C | | +42.62 | *A *C |
| | +1071.78 | *B *C | | −100.27 | *B *C |
| | +934.10 | *A *B *C | | −12.66 | *A *B *C |
| Final Equation in Terms of Actual Factors: | | | Final Equation in Terms of Actual Factors: | | |
| TGF-β = | +17305.75888 | | PDGF = | +985.97386 | |
| | +4.11786 | *collagen | | −0.11460 | *collagen |
| | +51.40516 | *thrombin | | +22.06084 | *thrombin |
| | −1017.56849 | *CaCl2 | | +17.76697 | *CaCl2 |
| | −1.00819 | *collagen *thrombin | | −1.54983E−003 | *collagen *thrombin |
| | −0.38912 | *collagen *CaCl2 | | +0.026082 | *collagen *CaCl2 |
| | +5.86709 | *thrombin *CaCl2 | | −4.31333 | *thrombin *CaCl2 |
| | +0.093188 | *collagen *thrombin *CaCl2 | | −1.26349E−003 | *collagen *thrombin *CaCl2 |
| IGF | | | KGF | | |
| Final Equation in Terms of Coded Factors: | | | Final Equation in Terms of Coded Factors: | | |
| IGF = | +31.82 | | KGF = | +79.79 | |
| | +0.29 | *A | | −3.11 | *A |
| | +0.45 | *B | | −2.21 | *B |
| | +0.019 | *C | | +1.98 | *C |
| | +0.045 | *A *B | | −0.93 | *A *B |
| | −0.96 | *A *C | | −0.36 | *A *C |
| | −1.22 | *B *C | | −1.53 | *B *C |
| | −0.068 | *A *B *C | | +3.96 | *A *B *C |
| Final Equation in Terms of Actual Factors: | | | Final Equation in Terms of Actual Factors: | | |
| IGF = | +28.07329 | | KGF = | +73.30599 | |
| | +2.65071E−003 | *collagen | | +8.86050E−003 | *collagen |
| | +0.40391 | *thrombin | | +1.23344 | *thrombin |
| | +0.53529 | *CaCl2 | | +2.03511 | *CaCl2 |
| | +5.72503E−005 | *collagen *thrombin | | −2.59146E−003 | *collagen *thrombin |
| | −3.94605E−004 | *collagen *CaCl2 | | −2.33493E−003 | *collagen *CaCl2 |
| | −0.056917 | *thrombin *CaCl2 | | −0.27497 | *thrombin *CaCl2 |
| | −6.76019E−006 | *collagen *thrombin *CaCl2 | | +3.94895E−004 | *collagen *thrombin *CaCl2 |

### Example 4

### Heavy Metal Analysis:

1. Samples were added with same amount of nitric acid and hydrogen peroxide solution (2:1) and then heated to 400° C. high temperature for the degradation process. The product after degradation was re-suspended into 0.2M nitric acid. Ion contents were examined by Inductively Coupled Plasma Mass Spectrometer (ICP-MS, SCIEX ELAN 5000, Perkin Elmer, USA). Results (Table 4) indicated that there were no significant difference in metal ions such as cobalt, cadmium, mercury and lead. The contents of heavy metals in animal body were not harmful. At the same time, contents of selenium were higher in all three kinds of P-BP then in FBS. Selenium is a kind of essential elements which contributes to the antioxidant capacity of cells.

TABLE 4
The ion contents in P-BP and commercial
serum was measured by ICP-MS

| | Co | Zn | Se | Cd | Hg | Pb |
|---|---|---|---|---|---|---|
| | (ppb) | | | | | |
| FBS | 0.261 | 133.8 | 0.950 | 0.048 | ND | 0.613 |
| Co+T+Ca− | 0.221 | 67.32 | 3.580 | 0.085 | 0.005 | 1.828 |
| Co+T−Ca− | 0.166 | 57.66 | 3.044 | 0.061 | ND | 1.576 |
| Co−T+Ca− | 0.259 | 42.87 | 3.851 | 0.072 | 0.011 | 1.824 |

### Example 5

### Examination of Fibronectin by Capillary Electrophoresis:

1. Capillaries were activated with 0.1N NaOH.
2. NaOH was washed out with DDH₂O.
3. Buffers were injected into the capillaries.
4. Samples were injected into the capillaries.
5. Electrophoresis was separated at under 25 KV.
6. After the separation, capillaries were washed by DDH₂O.
7. Conditions for the processes of capillary electrophoresis were presented in Table 5.
8. Buffer systems: Tricine—0.33% Tris, 1.44% glycine, and 0.1% SDS (pH=8.3) in 1000 mL DDH₂O.
9. The wave crest of fibronectin occurred in between 2-5 minutes (FIG. 9). Quantities of fibronectin were less in three kinds of P-BP then in commercial FBS. Fibronectin is a cell adhesion factor. Its quantities may affect cell adhesion rate within a certain time.

TABLE 5
Conditions for the processes of capillary electrophoresis

| | Time [min] | Item | Value | Period |
|---|---|---|---|---|
| 1 | | Washing pressure | 30.0 psi | 3.00 min |
| 2 | | Washing pressure | 35.0 psi | 2.00 min |
| 3 | | Washing pressure | 30.0 psi | 2.00 min |
| 4 | | Injection pressure | 1.5 psi | 20 sec |
| 5 | 0.00 | Isolated voltage | 25.0 KV | 15.0 min |
| 6 | 0.50 | Return-to-zero | | |
| 7 | 15.00 | Termination data | | |
| 8 | 15.00 | Washing pressure | 40 psi | 2.00 min |
| 9 | 18.00 | Finish | | |

### Example 6

### Isolation of Stem Cell:

1. All of the processes about sample collecting were in accordance with the ethical criterion of Chinese Culture University and Taipei Veterans General Hospital.
2. Fat samples were obtained from Taipei Veterans General Hospital. PBS with 5% antibiotics was added. Samples were then washed and separated from excess impurities through the process of centrifugation.
3. After 0.075% collagenase was added, degradation occurred upon incubation at 37° C. for 30 min.
4.10% FBS was added to stop the reaction. The precipitated stem cells were then isolated through centrifugation.
5. After filtering with 20-100 μm membrane, cells were cultured in flasks.

### Example 7

### Preparation of Human Mesenchymal Stem Cell Culture Medium:

1. DMEM powder medium was prepared in sterilized water with 1% antibiotics. 10% of commercial FBS (Hyclone, USA), serum free media (SFM; StemPro, USA), SFM (CEF, USA), and three groups of P-BP: Co+T+Ca−, Co+T−Ca− and Co−T+Ca− were filtered with 0.10-0.50 μm membrane and supplemented into the human mesenchymal stem cell culture medium.

### Example 8

### Culture of Human Mesenchymal Stem Cell:

1. Culture medium with P-BP, culture medium with FBS, and two kinds of commercial SFM (Invitrogen, USA, California; Cellular Engineering Technologies Inc., CET, USA, Iowa) were applied to compared the effect to human mesenchymal stem cell maintenance. Cells were seeded into 75T flasks (Orange Scientific) at 1×10⁴ cells/cm² and were incubated at 37° C. and 5% CO₂.

### Example 9

### Cell Adhesion Assay:

1. Medium with P-BP, medium with FBS and commercial serum free medium were added to the cultured stem cells.
2. Human stem cells were cultured for 24 hours and further stained by Trypan Blue. The adhesion and non-adhesion cells were then counted with cell counter (Invitrogen, USA).
3. Results of the calculated cell adhesion rate were represented as FIG. 10. Calculation formula of adhesion rate:

Numbers of adhesion cells/Numbers of adhesion and non-adhesion cells×100%

4. Wherein the optimal adhesion rate is the FBS-adhesion rate 85%. The results of fibronectin contents test showed higher quantities of fibronectin in group FBS.
In addition, fibronectin is a cell adhesion factor. Thus, results of the two kinds of tests proved consistent.

### Example 10

### Cell Proliferation Assay:

1. Medium with P-BP, medium with FBS and commercial serum free medium were added to the cultured stem cells.
2. Human stem cells were cultured for 24, 48, 72, and 96 hours, and further stained by Trypan Blue. The adhesion cells were then counted with cell counter (Invitrogen, USA) to determining the proliferation rate (FIG. 11).
3. The results indicated that all three kinds of P-BP exhibited a better proliferation rate in cultured human stem cells:

Co+T+Ca−: 221%

Co+T−Ca−: 253%

Co−T+Ca−: 164%

4. Comparing with commercial media, the present invention spends shorter time for cells to reach a certain numbers. In this case, large amount of cells can be expanded more efficiently to apply for other assays or medical use.

### Example 11

### The Cell Survival Rate Assay:

1. Medium with P-BP, medium with FBS and commercial serum free media were added to the cultured stem cells.
2. Human stem cells were cultured for 24, 48, and 96 hours, and further added with MTT reagent. Cells were then incubated under 37° C. for 3-5 hours.
3. DMSO was added to the cultured cells in order to dissolve the crystal. The absorbance was detected by spectrophotometer. The survival rate was then calculated (FIG. 12).
4. The results indicated that all three kinds of P-BP brings better survival rate of human stem cells than commercial FBS:

Co+T+Ca−: 160%

Co+T−Ca−: 176%

Co−T+Ca−: 174%

The results indicated that P-BP decrease the incidence of cell death more than commercial media. It can let the cells retain the original morphology and structure, and provide an optimum environment for human stem cell growth.

### Example 12

### Endotoxin Test:

Endotoxin test of samples were achieved by LAL kits (CAPE COD, US). 0.2 mL of each P-BP sample was added to the testing tubes of positive control and others. If gelation of the liquid occurred in the testing tubes, it means the concentration of endotoxin in the sample is over 0.03 EU/mL.
Endotoxin test of all three kinds of P-BP: Co−T+Ca− [symbol] Co+T−Ca− and Co+T+Ca− were examined in the testing tubes of positive control and others. Gelation occurred if the concentration of endotoxin in the sample is over 0.03 EU/mL. The results showed that all three kinds of P-BP samples did not go through gelation (FIG. 13). It indicated that quantities of endotoxin did not reach 0.03 EU/mL. If large amount of endotoxin exist in culture medium, it would affect cell growth even caused cell apoptosis.

### Example 13

### Trypsin Inhibition Assay:

FBS is generally applied for enzyme inhibition while cells are suspended by trypsin. This assay evaluates whether P-BP also inhibit trypsin activity. Its feasibility of replacing FBS is also estimated.
a. 1000 μg/ml collagen was placed into testing tubes. Trypsin was added to react with collagen. Medium with FBS and P-BP were then added to each mixture under 37° C., for the purpose of trypsin inhibition.
b. 20% TCA solution was added in order to precipitate the protein.
c. Samples were filtered by filter-paper No. 5. Concentration of 4-Hyproline in the filtrate were examined for protein degradation calculation.

Results of enzyme inhibition (FIG. 14) showed that there were no significant differences between P-BP and FBS in the inhibition of trypsin activity (p<0.05). It indicated that P-BP is able to apply for enzyme inhibition while cells are suspended by trypsin. P-BP efficiently inhibits trypsin activity thus avoids cell damage caused by continuous enzyme reaction.

While the invention has been described and exemplified in sufficient detail, for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

### BRIEF DESCRIPTION OF THE DRAWINGS

FIG. 1 shows the TGF-β's RSM.

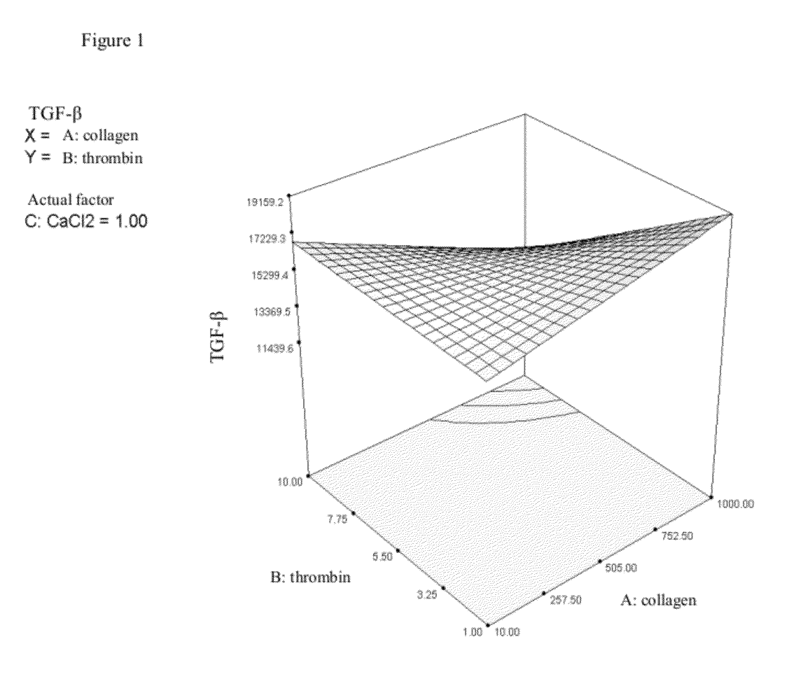

FIG. 2 shows the PDGF's RSM.

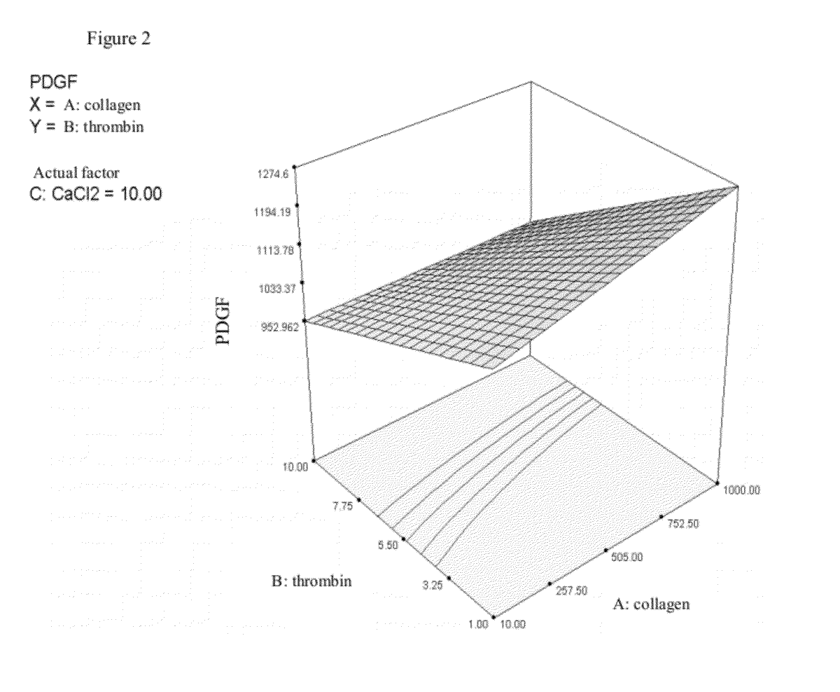

FIG. 3 shows the IGF's RSM.

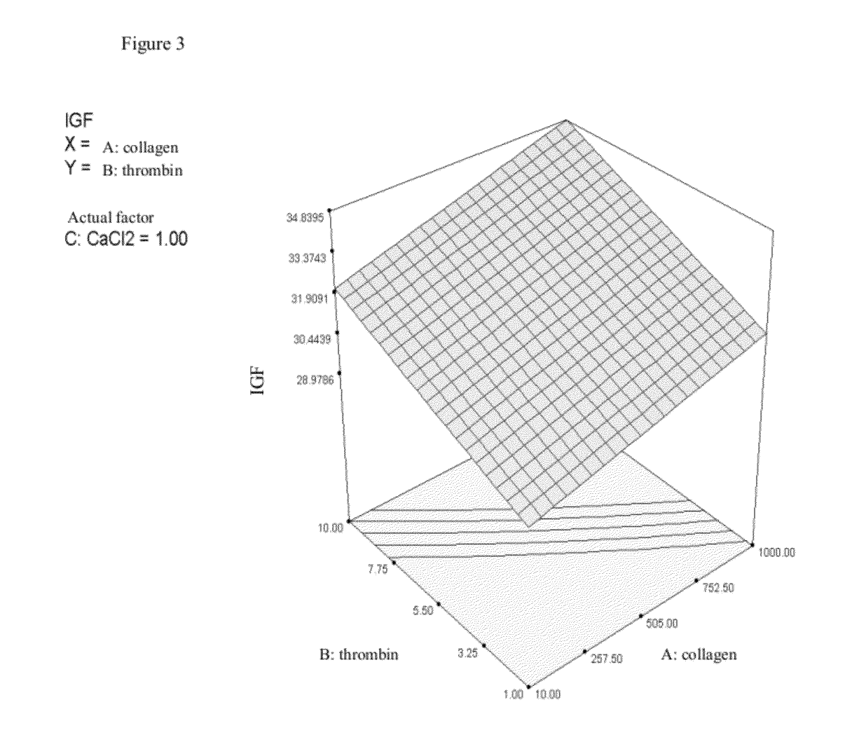

FIG. 4 shows the KGF's RSM.

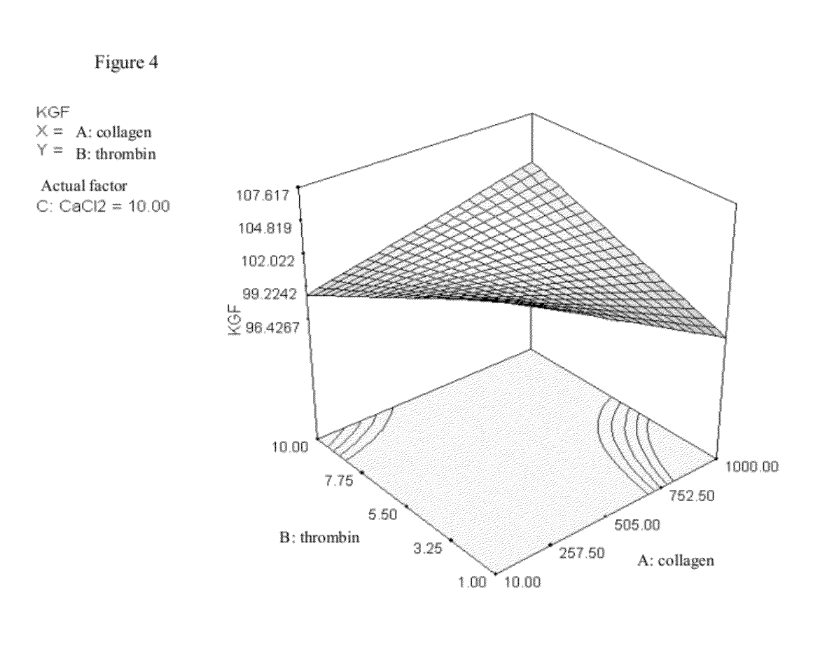

FIG. 5 shows the TGF-β's Cube.

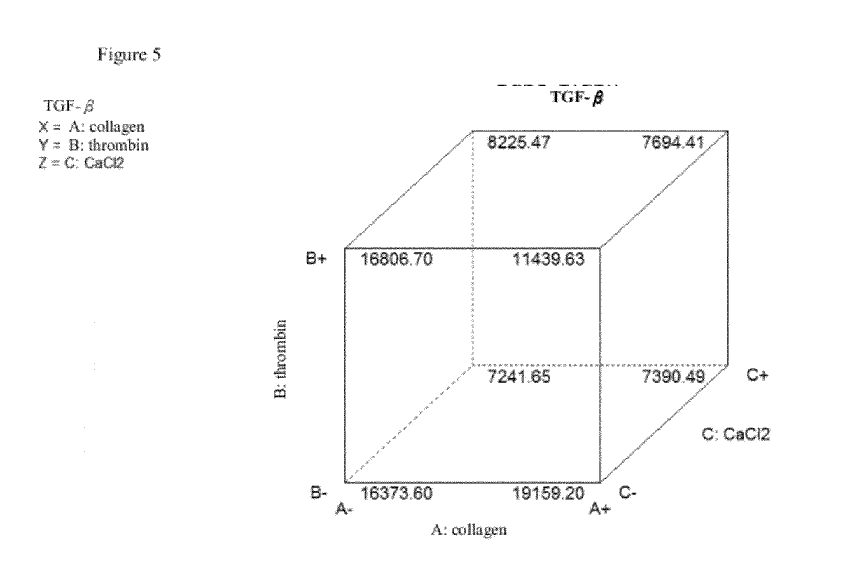

FIG. 6 shows the PDGF's Cube.

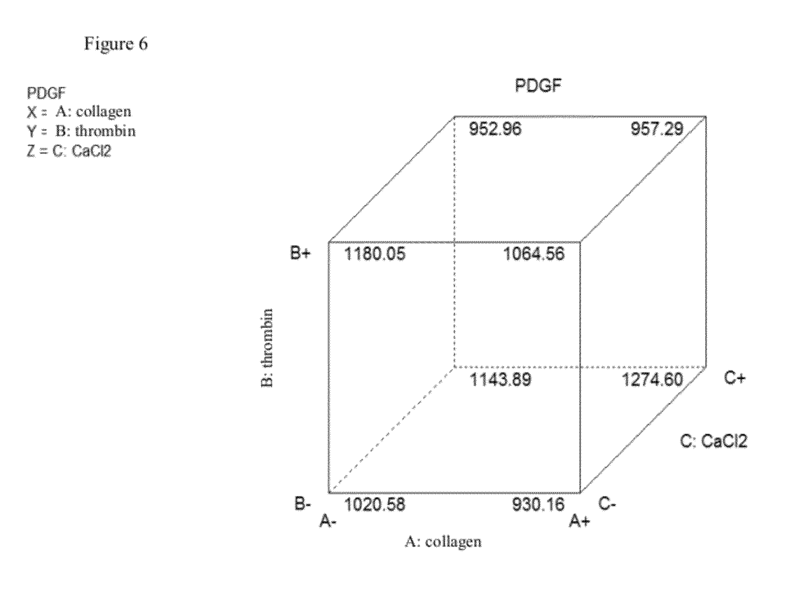

FIG. 7 shows the IGF's Cube.

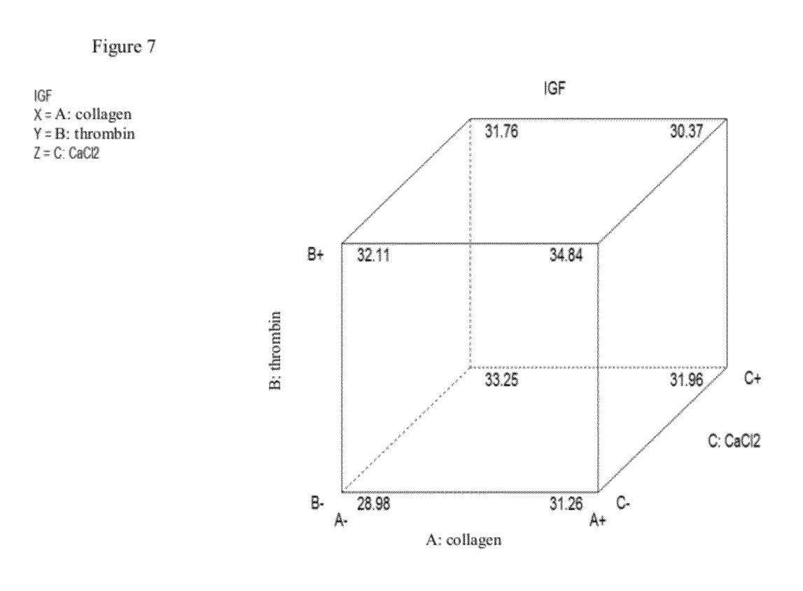

FIG. 8 shows the KGF's Cube.

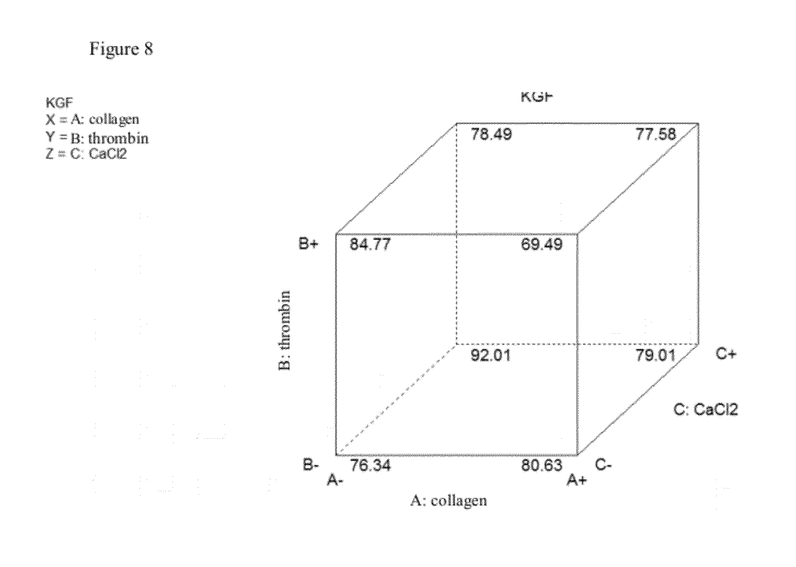

FIG. 9 shows the result of capillary electrophoresis.

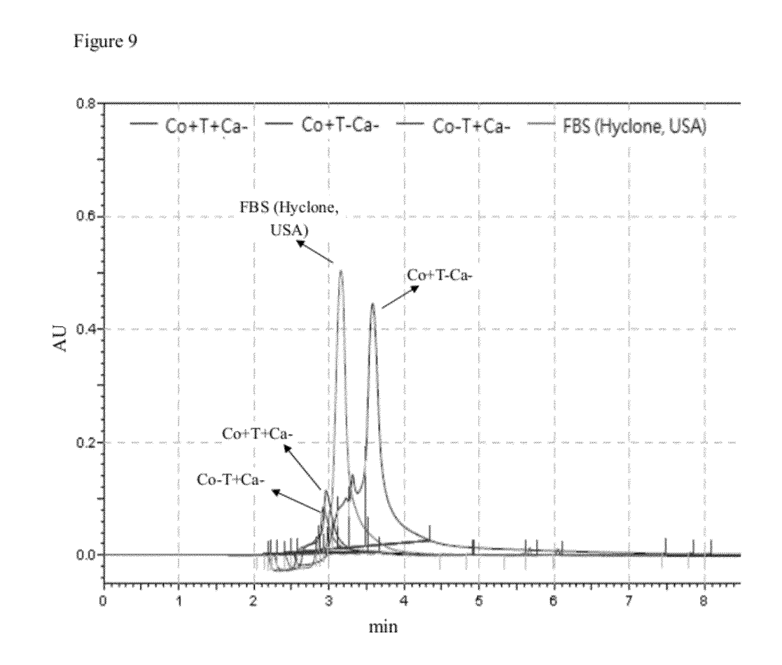

FIG. 10 shows the results of the calculated cell adhesion rate.

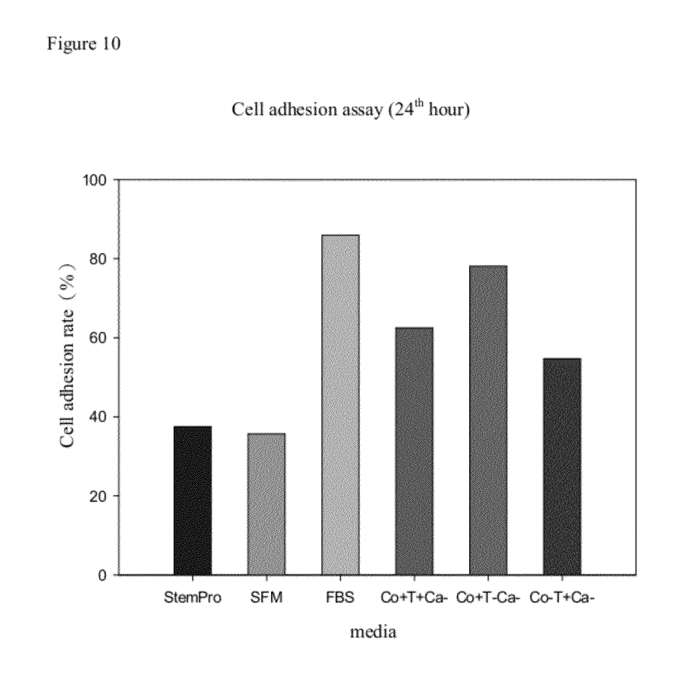

FIG. 11 shows the results of proliferation rate.

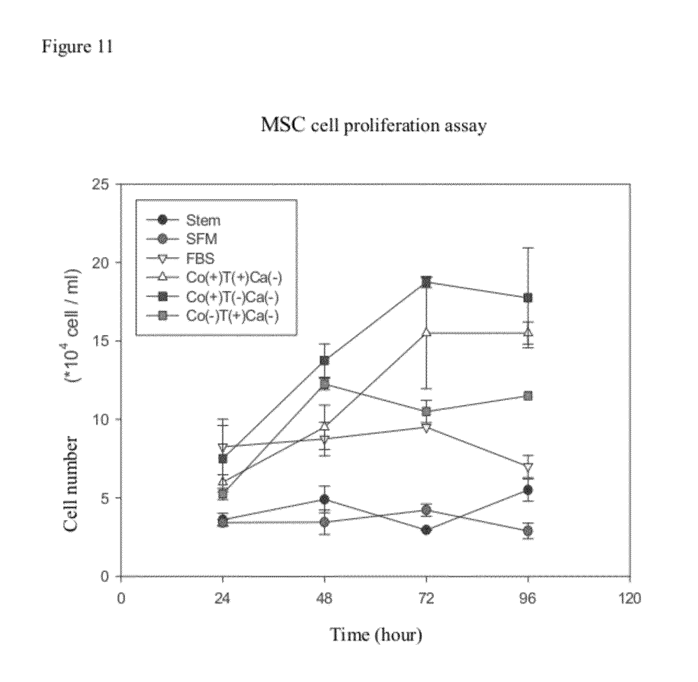

FIG. 12 shows the results of survival rate.

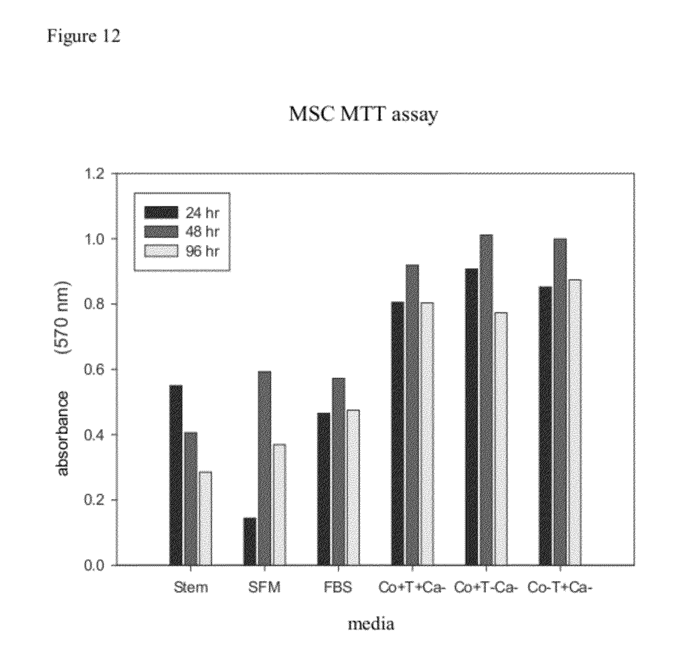

FIG. 13 shows the results of endotoxin test.

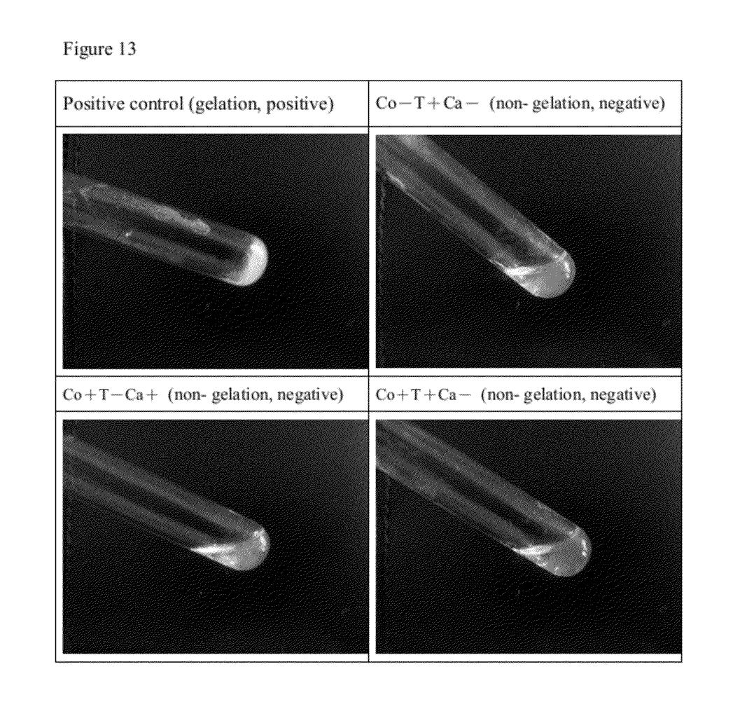

FIG. 14 shows the results of enzyme inhibition between P-BP and FBS in the inhibition of trypsin activity.

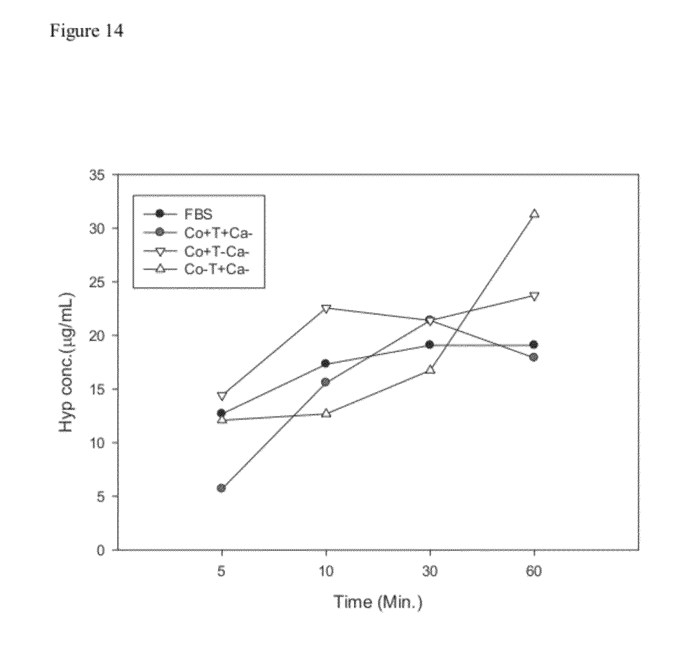

What is claimed is:

1. A method for making a supplement for cell cultivation media, comprising:
a. centrifuging mammal blood and collecting supernatant;
b. adding platelets activator into the supernatant to obtain activation supernatant;
c. sterilizing the activation supernatant.

2. The method of claim 1, wherein the centrifuging of step a means centrifuging at 100-5000 g for three times.

3. The method of claim 2, wherein the centrifuging of step a means centrifuging at 100-500 g for 5-10 minutes for the first time, centrifuging at 100-500 g for 5-10 minutes for the second time, and centrifuging at 1000-5000 g for 5-10 minutes for the third time.

4. The method of claim 1, wherein the supernatant of step a has platelet concentration from 50000-100000 platelets/ml.

5. The method of claim 1, wherein platelets activator contains collagen, thrombin and CaCl₂.

6. The method of claim 5, wherein the collagen, thrombin and CaCl₂ are in a range of concentration:
Collagen has concentration from 1000 μg/ml to 5000 μg/ml, thrombin has concentration from 5 U/ml to 50 U/ml, and CaCl₂ has concentration from 0.1% to 4.0%;
Collagen has concentration from 1000 μg/ml to 5000 μg/ml, thrombin has concentration from 0.1 U/ml to 2.0 U/ml, and CaCl₂ has concentration from 0.1% to 4.0%; or
Collagen has concentration from 10 μg/ml to 800 μg/ml, thrombin has concentration from 5 U/ml to 50 U/ml, and CaCl₂ has concentration from 0.1% to 4.0%.

7. The method of claim 1, wherein the cell cultivation media is stem cell cultivation media.

8. The method of claim 1, wherein the mammal is pig.

9. A supplement for cell cultivation media made by the method of claim 1.

10. The supplement of claim 9, which facilitates cell proliferation and cell survival.

11. The supplement of claim 9, which contains endotoxin less than 0.03 EU/mL.

12. The supplement of claim 9, which has inhibition activity of trypsin.

13. A cell cultivation media contains the supplement of claim 9.
